(19) United States
(12) Patent Application Publication
(10) Pub. No.: US 2024/0302467 A1
(43) Pub. Date: Sep. 12, 2024

(54) METHOD AND MRI SYSTEM FOR CALCULATING AT LEAST ONE OPTIMIZED INITIAL B1-SHIM FOR A MAGNETIC RESONANCE MEASUREMENT

(71) Applicant: Siemens Healthineers AG, Forchheim (DE)

(72) Inventors: Jürgen Herrler, Erlangen (DE); Patrick Liebig, Erlangen (DE)

(21) Appl. No.: 18/600,636

(22) Filed: Mar. 8, 2024

(30) Foreign Application Priority Data
Mar. 9, 2023 (CN) 202310226348.1

(51) Int. Cl. G01R 33/3875

(52) U.S. Cl. CPC G01R 33/3875

(57) ABSTRACT

A method for calculating a set of optimized initial B1-shims for an MR measurement is provided. A B1-shim includes a vector of complex B1-shim coefficients, each coefficient representing a scaling factor for one element of a multi-element transmit coil. The method includes receiving a set of previously measured B1-maps for one or more body parts of various test subjects, calculating a set of B1-shims for a plurality of different field-of-views in the one or more body parts using an optimization algorithm, and identifying which B1-shim has the best performance for a group of field-of-views using the previously measured B1-maps. The B1-shim is optimized for that group of field-of-views to obtain an optimized initial B1-shim.

This application claims the benefit of Chinese Patent Application No. CN 202310226348.1, filed on Mar. 9, 2023, which is hereby incorporated by reference in its entirety.

### BACKGROUND

In magnetic resonance imaging (MRI), at “ultra-high” magnetic field strengths of the BO-field (e.g., ultra-high field MRI, such as at more than 5T, at 7T or more), the radio frequency (RF) excitation field (e.g., B1-field) that is generated by the transmit RF coil becomes spatially strongly inhomogeneous, mainly due to the short Larmor wavelength. When using multi-element transmit coils (e.g., phased array RF coils), a technique referred to as “Parallel Transmit” (pTx) may be used to compensate for these inhomogeneities. A number of excitation coils are driven each with its own radio frequency (RF) pulse shape. The resulting excitation field arises from the interference of the fields generated by the individual coils and may thus be controlled in its spatial distribution.

Static pTx, also referred to as “B1-shimming”, consists of driving all coils with the same RF pulse shape (e.g., square or sinc), but scaling the RF pulse shape with coil-specific magnitudes and phases. This produces an excitation field that may be more spatially homogeneous than an uncalibrated excitation field. In 2D sequences, only one slice in the head is excited, and with adjustment of magnitudes and phases, significantly better field distributions may be generated especially in this case than in the uncalibrated case (e.g., circularly polarized B1-field, regardless of the patient or the layer to be excited).

To adjust these magnitudes and phases so that the resulting magnetization in the layer to be excited becomes as spatially homogeneous as possible, a non-convex optimization problem based on the B1-fields of the individual transmitting coils is to be solved. In addition, the B1-fields of the individual transmitting coils are dependent on the anatomy of the individual patient, which is why this adjustment may only be performed during the examination. Thus, the optimization is to be performed quickly but still function robustly and for a wide variety of B1-fields. To calculate the best possible combination of amplitudes and phases of the transmitting channels (e.g., “B1-shim”), a minimization problem is formulated that may, for example, be solved with gradient descent methods under certain constraints, such as maximum voltage at a transmitting channel, and limited Specific Absorption Rate (SAR) exposure. For example, inner-point methods, as described in K. Majewski “Simultaneous optimization of radio frequency and gradient waveforms with exact Hessians and slew rate constraints applied to kT-points excitation,” Journal of Magnetic Resonance, Volume 326, 2021, 106941, ISSN 1090-7807 or active-set methods, as described in A. Hoyos-Idrobo, P. Weiss, A. Massire, A. Amadon and N. Boulant, “On Variant Strategies to Solve the Magnitude Least Squares Optimization Problem in Parallel Transmission Pulse Design and Under Strict SAR and Power Constraints,” in IEEE Transaction on Medical Imaging, vol. 33, no. 3, pp. 739-748, March 2014) may be used. Further, a very successful and established algorithm was presented by Setsompopp et al. in K. Setsompop, L. L. Wald, V. Alagappan, B. A. Gagoski, E. Adalsteinsson “Magnitude least squares optimization for parallel radio frequency excitation design demonstrated at 7 Tesla with eight channels,” Magn. Reson. Med. 2008; 59(4):908-915.

However, all of these algorithms are by default initialized with a B1-shim having constant amplitudes and phases, which produce a circularly polarized B1 field. This often leads to suboptimal results, since the problem to be solved is non-convex. Thus, the optimization algorithm may end up not in the absolute minimum, but in a suboptimal local minimum.

A well-known problem is the appearance of “holes” in the resulting magnetization distributions (e.g., the resulting B1-field magnitude in a small local area within the imaging slice is zero or almost zero), so that the resulting MR signal is also zero or close to zero. This is particularly undesirable, since with zero signal amplitude, no diagnosis is possible in this area.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that allows improved B1-shimming, as well as a related computer program and magnetic resonance imaging system, are provided. As another example, an improved method for B1-shimming that reliably avoids the above-identified disadvantages of having “holes” in the magnetization distribution, and therefore in the magnetic resonance (MR) image, is provided.

According to a first aspect of the present embodiments, a method is provided for calculating at least one optimized initial B1-shim for a magnetic resonance measurement, where a B1-shim includes a vector of complex B1-shim coefficients, each coefficient representing a scaling factor for one element of a multi-element transmit coil that is to be used in the magnetic resonance measurement. The method includes: (a) receiving a set of previously measured B1-maps of the multi-element transmit coil for one or more body parts of various test subjects; (b) calculating a set of B1-shims for a plurality of different field-of-views in the one or more body parts of the various test subjects from the previously measured B1-maps using an optimization algorithm; (c) identifying which B1-shim has the best performance for a group of field-of-views by using the previously measured B1-maps; and (d) optimizing the B1-shim for that group of field-of-views to obtain an optimized initial B1-shim.

The present embodiments have recognized that better B1-shims may be reached by using better-fitting initial shims that are referred to as optimized initial B1-shims. The method has been devised to overcome the difficulty, that the optimization problem of B1-shimming is non-convex; thus, if the starting values are unsuitable, the optimization algorithm may end up in an unfavorable local minimum. Therefore, the present embodiments aim at using a favorable starting point for the B1-shimming optimization.

A method may be performed once for each multi-element transmit coil and/or for each magnetic resonance imaging system (e.g., the method may not have to be repeated for each MR measurement). The method may also be carried out only once by the manufacturer of the MRI system or the multi-element transmit coil, and the result (e.g., one or a set of optimized initial B1-shims) may be stored in a digital storage to which the MRI system has access. The method may be used for optimal B1-shimming within a certain field-of-view within a body part of a subject. However, the method may also be used to find optimal B1-shims (e.g., for RF saturation pulses that are to excite or suppress the spins outside of an imaging field-of-view).

According to an embodiment, a method is provided to select one of the sets of optimized initial B1-shims during each MR measurement. This optimized initial B1-shim may then be used as the starting point for the online-optimization (e.g., during the MR measurement). In this optimization, the specific absorption rate (SAR) limits for the complete magnetic resonance measurement (e.g., for the MR sequences to be used) may be taken into account. For example, a number of different optimized initial B1-shims may be used for different field-of-views.

In the following, a field-of-view may be a region of interest within a body part of the subject that is to be excited by an RF pulse (e.g., a particular slice or slab through the body part, which may be in sagittal, coronal or transversal orientation, or any orientations in between). The field-of-view may also be a part of such slice or slab (e.g., only the inner part) when the outer region is not of interest for the MR measurement. The MR measurement may be any magnetic resonance imaging or spectroscopy measurement that is to be carried out during an examination of a subject. The subject may be a human or animal subject (e.g., a patient). The body part may be any body part on which an MR measurement is to be carried out (e.g., a head, neck, a limb, part of a limb like a knee, hand, foot, thorax, abdomen, or parts thereof such as an inner organ, such as, the liver, heart, kidney, bowels, etc.).

A B1-shim, which may also be referred to as RF-shim or RF-shim setting, includes a vector of complex B1-shim coefficients, each coefficient representing a scaling factor for one element of a multi-element transmit coil that is to be used in the magnetic resonance measurement. This coefficient is usually complex, to take into the account not only the magnitude, but also the phase of the RF pulse. A multi-element transmit coil may be a local radio frequency (RF) coil that is placed close to the body part (e.g., a phased-array coil). The B1-shim is, for example, a static B1-shim. In other words, the B1-shim coefficients are used to scale the RF power delivered by each element of the RF coil, but each coil is driven with the same pulse shape.

The method includes a first act (a) of receiving a set of previously measured B1-maps of the multi-element transmit coil for one or more body parts of various test subjects. A B1-map, also referred to as B1 field sensitivity map, is thus the B1-field distribution of each element/channel of the transmit coil. Thus, for each body part in each test subject, as many B1-maps are measured as there are elements in the multi-element transmit coil. The B1 field distribution of all elements together are referred to as “B1-map” herein. Because the B1-field has magnitude and phase, the B1-field is a complex map, also referred to as B1+ map. The complex B1-maps may be obtained, for example, with a hybrid B1+ mapping technique as described in Van de Moortele P. F. et al. “Calibration tools for RF shim at very high field with multiple element RF Coils: from ultra fast local relative phase to absolute magnitude B1+ mapping,” Proceedings of the 15th Annual Meeting of ISMRM, Berlin, 2007, Abstract No. 1676. This method includes merging one absolute large flip angle |B1+| map measured with all coils transmitting together in a known B1 shim setting, with N_{Tx} complex, relative B1+ maps derived from small flip angle, multi-slice gradient echo images acquired with only one Tx element transmitting at a time. Other methods for obtaining subject-specific B1 field sensitivity maps are described, for example, in the Thesis by Kawin Setsompop “Design Algorithms for Parallel Transmission in Magnetic Resonance Imaging,” Massachusetts Institute of Technology, 2008, which is incorporated herein by reference.

The B1-shimming problem therefore essentially consists in finding the B1-shim, and thus the vector of complex B1-shim coefficients, which allows to combine the B1-fields of the number of elements of the RF coil, so that the overall B1-field is as homogeneous as possible. This optimization is done in act (b) and in a number of other method acts of various embodiments using an optimization algorithm. This optimization algorithm may use a static B1 shimming technique (e.g., the RF shim settings are constant during the RF pulse). A static RF shimming method is, for example, described in Mao W, Smith M B, Collins C M. “Exploring the limits of RF shimming for high-field MRI of the human head,” Magn. Reson. Med. 2006; 56(4): 918-922. With this technique, only the overall amplitude and phase, and not the shape of the RF pulse, are allowed to vary from transmit element to transmit element. In order to excite a slab, a sinc pulse with different amplitude and phase may be applied to each transmit element. The optimization algorithm may use a gradient descent method (e.g., an inner-point method), as disclosed in the above-cited article by Majewski. Circularly polarized B1-fields may be used as starting points for this optimization algorithm. However, other optimization algorithms known in the art may be used in this act.

The set of previously measured B1-maps may include either B1-maps of the same kind of body part (e.g., only of the head of various test subjects). The set of previously measured B1-maps may also include B1-maps of a variety of body parts of various test subjects (e.g., a variety including B1-maps measured for heads, shoulders, knees, thorax, etc.). The B1-maps have been measured on various test subjects using known methods of measuring the B1-maps of a multi-element transmit coil. Such measurements may be performed at the beginning of each MR measurement that requires B1-shimming.

Act (b) includes calculating a set of B1-shims for a plurality of different field-of-views, using the B1-maps for the one or more body parts of the various test subjects. The different field-of-views may, for example, correspond to different slices or slabs through the body part (e.g., a stack of slices in one particular orientation). This is done because the required B1-shim may be different for a different field-of-view within the same body part, because of the severe inhomogeneity of the B1-maps of each element of the transmit coil.

These B1-shims, although they have been optimized, may still not provide the ideal B1-shim for each field-of-view, for the reasons described above. Therefore, the method includes a further act (c) of identifying which B1-shim has the best performance for a group of field-of-views, by using the previously measured B1-maps. In this act, B1-shims that have been calculated for field-of-views in one body part (e.g., the head) may be applied to completely different body-parts to find out whether the B1 shims may also give satisfactory results. A performance index may then be calculated, and groups of field-of-views with similar performance may be formed. This may be done by a clustering method. It is advantageous to find B1-shims that perform well for a group of field-of-views in order to find a set of potentially favorable initial B1-shims that may be used as a starting point for an optimization algorithm during an actual MR measurement. To further improve the B1-shim, which has been identified as having the best performance for a group of field-of-views, the B1-shim is optimized for that group of field-of-views to obtain an optimized initial B1-shim. Also, in this act (d), an optimization algorithm using a gradient descent method (e.g., an inner-point method as disclosed by Majewski) or a B1-shimming method as described in Mao W et al. may be used.

According to an embodiment, the method includes the acts of: (c1) clustering the B1-shims calculated for the plurality of different field-of-views and identifying an average B1-shim for each cluster; (c2) applying the average B1-shims to each of the plurality of field-of-views and calculating a performance index for each combination of average B1-shim and field-of-view using the previously measured B1-maps; (c3) clustering the field-of-views according to their performance indices to identify groups of field-of-views that perform similarly well with a similar B1-shim; and (d) for each group of field-of-views, optimizing the B1-shim to obtain an optimized initial B1-shim.

The B1-shims that have been optimized for the plurality of different field-of-views may be clustered according to their similarity, so that B1-shims that have similar coefficients form one cluster. Once the clusters have been defined, the B1-shims of each cluster may be averaged to calculate an average B1-shim. The average may, for example, be the arithmetic mean, the median, or any other suitable type of average for the cluster. The average B1-shim may also be the midpoint or center of gravity of each cluster. The idea behind act c1 is that the average B1-shims together define a space where possible suitable shims may be found.

In a next act c2, these average B1-shims are then applied to each field-of-view of the plurality of field-of-views, and a performance index is calculated for each combination of average B1-shim and field-of-view, using the previously measured B1-maps for each field-of-view in a body part. Thereby, B1-shims that have been optimized for one subject may now be used on the same or a different field-of-view in another subject. To keep the calculation effort in this step within a certain limit, it is helpful to only apply the average B1-shims. In this act, “apply” may provide that the average B1-shim is used as the starting point of an individual optimization for that field-of-view. The performance index indicates whether the average B1-shim results in a homogeneous magnetization distribution when applied to the particular field-of-view. The performance index may include a comparison of a target flip angle with an actual flip angle that will be achieved when using the B1-shim with the previously measured B1-maps.

In a next act c3, also the field-of-views are clustered according to their performance indices in order to identify groups of field-of-views that perform similarly well with a similar B1-shim. The clustering may identify groups of field-of-views, where not all field-of-views are part of a group, but only those that are close to the average or center of gravity of each cluster.

Once these clusters have been identified, a B1-shim is optimized for each cluster or group of field-of-views to obtain an optimized initial B1-shim. In a useful embodiment, this optimization uses a number of different B1-shims as staring point (e.g., the B1-shims that have been optimized for the different field-of-views in act (b)) and evaluating the performance of each resulting B1-shim (e.g., by comparing the resulting magnetization distribution with the target magnetization distribution in the group of field-of-views). The resulting B1-shim that has the best performance for that group of field-of-views is then taken as an “optimized initial B1-shims”.

The different clusters in the second feature space represent different B1s, for which the MR system is to be able to perform robust B1-shimming. By optimizing the B1-shim for a number of field-of-views at a time, more robust shims may be found than for an individual optimization, in which, for example, the above-mentioned “holes” may be produced. Therefore, one may also use the method of the present embodiments to create B1-shimming for universal pulses (e.g., saturation pulses).

According to an embodiment, the acts of performing a clustering of the B1-shims and identifying an average B1-shim for each cluster may be performed by (c1.1) representing the set of B1-shims calculated in act (b) in a first feature space, where the dimensions of the first feature space are the complex shim coefficients of each B1-shim, (c1.2) performing a cluster analysis of the B1-shims in the first feature space and calculating a midpoint of each cluster, each midpoint being the average B1-shim for that cluster.

Thus, the feature space in which the set of B1-shims are arranged may have twice as many dimensions as there are elements in the multi-element RF coil, or as the number of transmit channels of the RF coil. In this first feature space, each B1-shim is represented by one data point. The clustering may be performed using a known cluster analysis algorithm, such as k-means clustering. According to an embodiment, a useful number of clusters may be found by calculating the within-cluster-sum-of-squares (WCSS) value as a function of the number of clusters/classes of shims. According to an embodiment, between 20 and 1000 (e.g., between 40 and 500 or between 60 and 200 B1-shims) may be calculated in act b (e.g., so many different field-of-views in the one or more body parts of the various test subjects may be used). The WCSS value is a measure for how similar the B1-shims within one cluster are. If the WCSS value has a sharp drop at a certain number of clusters, then this number of clusters may be suitable. However, if there is no such sharp drop, the user may have to choose the number of clusters, or the number of clusters may be predetermined. For example, it may be determined that between 5 and 30 (e.g., between 10 and 20) B1-shims are to form one cluster. Alternatively, one may also determine the number of clusters that are desirable for the different field-of-views (e.g., between 10 and 40 or between 20 and 30 clusters).

In a next act, the midpoint of each cluster is to be determined. This may be the center of gravity of each cluster (e.g., the arithmetic mean or any other kind of mean). The midpoint or center of gravity is then defined as the average B1-shim for that cluster.

According to an embodiment, the acts of clustering the field-of-views according to their performance indices and optimizing the B1-shim to obtain an optimized initial B1-shim for this group of field-of-views are performed by: (c3.1) representing the performance indices calculated in act (c2) in a second feature space, in which each field-of-view is represented by one data point, and the dimensions of the feature space are the performance indices of each average B1-shim; (c3.2) performing a cluster analysis on the second feature space, and determining the group of field-of-views which are closest to the center of each cluster; (d.1) for each group of field-of-views, calculating an optimized B1-shim using an optimization algorithm; and (d.2) providing the optimized B1-shims as optimized initial B1-shims for the field-of-views within the group of field-of-views.

Thus, the performance indices calculated for each field-of-view are represented in a second feature space, where dimensions of the second feature space are the performance indices of each average B1-shim. Thus, each field-of-view is represented by one data point. The performance index may be the root-mean-square-error (RMSE) (e.g., a value obtained by simulating a flip angle distribution or magnetization distribution for each voxel in the field-of-view from the B1-maps and the average B1-shim, comparing this value for each voxel with the target flip angle or target magnetization, taking the square of the deviation, and averaging this square for all voxels within the field-of-view). This value may be divided by the square of the target flip angle or target magnetization, respectively, in order to obtain a normalized root-mean-square-error, NRMSE. This value is the performance index of each combination of field-of-view and B1-shim. The respective feature space thus has a number of dimensions that is the same as the number of average B1-shims.

In a next act, a cluster analysis may be performed on the second feature space in order to determine the group of field-of-views that are closest to the center of each cluster. The clustering may again be performed using k-means clustering. Thereby, the different field-of-views may be classified into different clusters. Thereby, those field-of-views or slices that perform similarly well for similar shims may be identified. As for the first feature space, the WCSS values may be calculated for different numbers of clusters, and a suitable number may thereby be identified. Alternatively, the number of clusters may be pre-determined (e.g., that between 10 and 50 or between 15 and 30 field-of-views should be within each cluster).

By performing the clustering in the second feature space, one may find out which field-of-views may advantageously be grouped together in order to find one suitable initial B1-shim.

Finally, the optimized initial B1-shims are now optimized for each cluster. According to an embodiment, the B1-shim is only optimized for those field-of-views that are closest to the center of gravity of each cluster, whereby, for example, the Euclidean distance may be used. The center of gravity of each cluster may be calculated as described above (e.g., by calculating the arithmetic mean). Different clusters represent different B1 field distributions, for which one is to optimize during the MR measurement.

All method acts described so far may be performed offline (e.g., using previously measured B1-maps that may have been obtained from a variety of test subjects at an earlier time). Therefore, the method according to the first aspect of the present embodiments is computer-implemented and may be performed on any kind of computer or processing unit (e.g., on a CPU or GPU of a computer, such as a PC, laptop, smartphone, tablet computer, cloud computer, etc.).

According to an embodiment, the clustering of the B1-shims in the first feature space and/or the clustering of the field-of-views in the second feature space may be performed using a k-means clustering algorithm. Such a clustering algorithm is, for example, described in “https://de.wikipedia.org/wiki/k-means_clustering.”. Such a clustering algorithm is a method of vector quantization that aims to partition n observations into k clusters, in which each observation belongs to the cluster with the nearest mean, also referred to as cluster center.

According to an embodiment, the performance index for each combination of B1-shim and field-of-view may be done by simulating a magnetization distribution or flip angle distribution from each B1-shim in the field-of-view, and comparing the simulating distribution with a target magnetization or flip angle distribution, respectively (e.g., by calculating a root-mean-square deviation, as described above). The magnetization distribution may be the transversal magnetization that would be generated when applying each B1-shim to the respective field-of-view with its respective B1-maps. The magnetization may be complex (e.g., having a magnitude and phase). Instead of the magnetization, one may also work with the flip angle distribution, where the flip angle may be a real value for each pixel or voxel within the field-of-view.

According to an embodiment, the method includes calculating a set of optimized initial B1-shims (e.g., one for each group of field-of-views mentioned in acts (c) and (d)). For example, the method may be used to calculate a set of 10 to 80 or 20 to 60 optimized initial B1-shims. Thereby, one has a selection of different starting points for the B1-shimming during the MR measurement.

The method also includes a method of finding the ideal starting point for B1-shimming during a MR measurement, from the set of optimized initial B1-shims. In one embodiment, this selection method uses the small angle approximation for all flip angles. Thereby, the calculation is simplified, but the calculation is sufficiently precise to find differences between the various initial B1-shims and allows to define a matrix for each field-of-view, and to estimate the magnetization distribution for the various optimized initial B1-shims using a simple matrix multiplication, as described in the following in more detail.

For example, the method may include a shimming method for performing B1-shimming during a magnetic resonance measurement on a field-of-view within the body part of a subject using a multi-element transmit coil. The shimming method includes: (i) receiving a set of optimized initial B1-shims that have been calculated by a method of the present embodiments; (ii) measuring B1-maps of the body part; (iii) using the B1-maps for calculating the magnetization distribution resulting from the combination of each of the set of optimized initial B1-shims with the field-of-view and storing the result in a magnetization matrix; (iv) for each of the set of optimized initial B1-shims, calculating a term including a parameter including a comparison of the magnetization matrix with a target magnetization distribution, a parameter including the minimal magnetization or flip angle within the magnetization matrix, and optionally a parameter including the phase rotation of the magnetization matrix; and (v) selecting the optimized initial B1-shim for which the term calculated in act (ii) is at an extremum, as the starting point of a B1-shimming optimization.

This part of the method may be performed online (e.g., once the subject or patient has been positioned within the MRI system because it requires B1-maps of the body part on which the MR measurement is to be performed). These B1-maps are then used for calculating the magnetization distribution resulting from the combination of each of the set of optimized initial B1-shims with the field-of-view, which is to be measured, and storing the results in a magnetization matrix. The “magnetization” may be the complex transverse magnetization that would be obtained when applying a certain RF pulse using the optimized initial B1-shim. Alternatively, the “magnetization” may also be the flip angle, or alternatively the B1-field, that is obtained when using the optimized initial B1-shim on the multi-element transmit coil from which the B1-maps have been measured. Magnetization, flip angle, and B1-field all describe the same effect; therefore, in this text, the “magnetization distribution” may also be the flip angle distribution or the B1-field distribution generated by the multi-element transmit coil. This magnetization distribution is stored in a magnetization matrix in act (iii) for each optimized initial B1-shim. The “matrix” may be that each pixel in the field-of-view is assigned one element in the magnetization matrix. The pixel size within the magnetization matrix may be as large as in a MR image. Alternatively, the pixel size may be somewhat larger in order to simplify the calculation (e.g., using a pixel size of 2 to 4 mm). These matrices may then be used to calculate a term including a comparison of the magnetization matrix with a target magnetization distribution (or target flip angle distribution), the minimal flip angle within the magnetization matrix, and optionally the phase rotation of the magnetization matrix.

The comparison of the magnetization matrix with a target magnetization distribution may include calculating the root-means-square deviation (RMSE) between the magnetization matrix and the target magnetization distribution, as described herein. For example, the corresponding parameter may be the normalized RMSE. Evidently, this RMSE may be as small as possible in order to find the best starting point among the set of optimized initial B1-shims. Further, the term may include the minimum flip angle within the magnetization matrix, but in a way, the optimized initial B1-shims may be preferred, where the minimal flip angle is as large as possible. The minimal flip angle within the magnetization matrix characterizes possible “holes” in the magnetization distribution and thus in the final MR image. Therefore, magnetization matrices that have some local areas where the magnetization or flip angle is very small are a less preferred starting point. For example, the parameter may include one minus the normalized minimal magnetization. According to an embodiment, the minimal magnetization matrix excludes up to 5% up to 2%, or up to 1% of the magnetizations in order to avoid possible outliers. As an optional third parameter, the term may include a parameter including the phase rotation of the magnetization matrix. This may be calculated using the curl of the vector field that represents the magnetization matrix.

This term is calculated for each of the set of optimized initial B1-shims, and that B1-shim for which the term is at an extremum (e.g., at a minimum) is selected as the starting point of a B1-shimming optimization. This B1-shimming may then be performed again using a known optimization algorithm (e.g., a gradient descent method such as the inner points method). However, by using a very good initial B1-shim, it has been found that the B1-shimming optimization produces much more robust results than the state of the art. Further, the shimming method may be performed very quickly (e.g., in less than 1 second) during the MR examination while the patient is in the MR scanner. Time is very important during the MR examination itself.

According to an embodiment, the term of which the extremum (e.g., the minimum) is taken includes the root-mean-square deviation between the magnetization matrix and a target magnetization distribution. The RMSE may, for example, be the NRMSE. The RMSE may be weighted in the term.

Also, the minimum magnetization within the magnetization matrix may be weighted by a pre-determined weight, as may be the term including the phase rotation of the magnetization matrix. Thus, the term calculated in act (iv) may be a weighted sum of a parameter including the root-mean-square deviation between the magnetization distribution and a target magnetization distribution, a term including the minimum flip angle within the magnetization matrix, and optionally a term including the phase rotation of the magnetization matrix.

The present embodiments are also directed to a computer program including a program code that causes a computer to carry out the method according to an embodiment when the computer program is executed on a computer. The computer may be a stand-alone computer or a processing unit, such as a CPU, GPU, that may be part of a PC, main frame computer, cloud computer, server, or part of a control computer of an MR system. This is, for example, true for the shimming method, which may be carried out during an MR examination.

According to a further aspect, the present embodiments relate to a non-transient computer readable medium including a computer program with instructions that, when carried out on a computer, will cause the computer to carry out the method according to an embodiment. The computer-readable medium may be any digital storage medium (e.g., an optical or magnetic storage medium) or a solid-state digital storage medium, such as an SD-card, SSD-card, USB-stick, hard disc or cloud solution.

The present embodiments are further directed to a B1-shim design unit configured for calculating at least one optimized initial B1-shim for a magnetic resonance imaging measurement on a field-of-view within a body part of a subject, where a B1-shim includes a vector of complex B1-shim coefficients. Each coefficient represents a scaling factor for one element of a multi-element transmit coil that is to be used in the magnetic resonance measurement. The B1-shim design unit includes: a data interface configured for receiving a set of previously measured B1-maps of the multi-element transmit coil for one or more body parts of various test subjects, and for outputting the optimized initial B1-shim; and a calculating unit configured for performing the method of calculating at least one optimized initial B1-shim according to an embodiment. For example, the B1 shim design unit is configured to carry out the method according to the present embodiments.

The B1-shim design unit may operate offline (e.g., the B1-shim design unit does not have to be in direct connection to an MRI system), since the B1-shim design unit requires only B1-maps measured previously from one or more (different) body parts of various test subjects. The rest of the method for calculating one or a set of optimized initial B1-shims is performed by simulation, as described herein. The output is at least one optimized initial B1-shim. Such initial B1-shim may be used in the shimming method, or may be used by a control unit for a magnetic resonance imaging system, which is also part of the present embodiments. The control unit is configured to carry out at least the B1-shimming method described herein. According to an embodiment, the control unit may also be configured to calculate the at least one optimized initial B1-shim (e.g., may be the B1-shim design unit). Both the B1-shim design unit and the control unit may include a processing unit (e.g., a CPU or a gradient coil system).

Finally, the present embodiments are directed to an MRI system including such a control unit. The MRI system may further include a main magnet, a gradient coil system, and a multi-element transmit coil, as well as other elements known in the art.

All embodiments, features, and advantages of the method according to the present embodiments also apply to the computer program, the non-transient computer readable medium, the B1-shim design unit, the control unit, and the MRI system of the invention, and vice versa.

### BRIEF DESCRIPTION OF THE DRAWINGS

FIG. 1 is a schematic representation of a magnetic resonance imaging (MRI) apparatus according to an embodiment;

FIG. 2 shows a B1-map of a multi-element RF coil having six channels;

FIG. 3 shows a schematic cross section through the human head, demonstrating the position of six field-of-views;

FIG. 4 is a flow diagram of a method according to an embodiment; and

FIG. 5 is a flow diagram of a method for selecting a suitable optimized initial B1-shim during an MR measurement.

Similar elements are designated with the same reference signs in the drawings.

### DETAILED DESCRIPTION

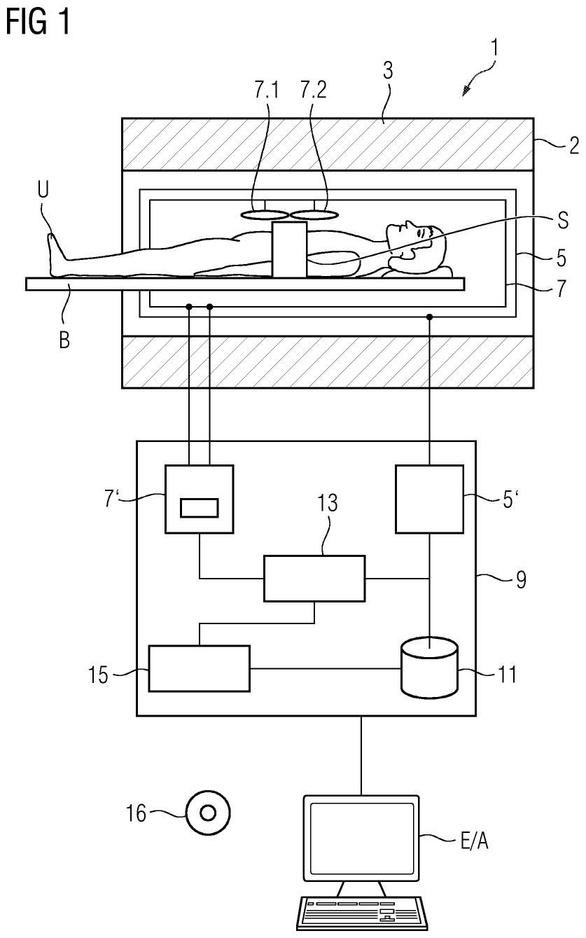

FIG. 1 schematically shows a magnetic resonance (MR) apparatus 1 according to an embodiment. The MR apparatus 1 has an MR data acquisition scanner 2 with a magnet 3 that generates a constant magnetic field (B0-field), a gradient coil arrangement 5 that generates gradient fields, one or more radio-frequency (RF) antennas 7 for transmitting and receiving RF signals (e.g., B1-fields), and a control computer 9 configured to perform a method of the present embodiments. The one or more RF antennas 7 may include a multi-element RF coil 7, such as a multi-channel coil array, including at least two coils (e.g., the schematically shown coils 7.1 and 7.2) that may be configured to transmit and/or receive RF signals (e.g., MR signals). In order to acquire MR data from an examination subject U (e.g., a patient or a phantom), the examination subject U is introduced on a bed B into the measurement volume of the scanner 2. The slab S is an example of a field-of-view within a body part of the examination subject, from which MR data may be acquired using a method according to an embodiment of the present embodiments. The control computer 9 controls the MR apparatus 1, and may control the gradient coil arrangement 5 with a gradient controller 5′ and the RF antenna 7 with a RF transmit/receive controller 7′. The RF antenna 7 has multiple channels corresponding to the multiple coils 7.1, 7.2 of the coil arrays, in which signals may be transmitted or received. The control computer 9 also has an imaging protocol processor 15 that determines the imaging protocols, including the measurement of B1 maps of the multi-element RF coil 7. A control unit 13 of the control computer 9 is configured to execute all the controls and computation operations required for acquisitions. Intermediate results and final results required for this purpose or determined in the process may be stored in a memory 11 of the control computer 9. Also, the at least one optimized initial B1-shim may be stored in the memory 11. A user may enter control commands and/or view displayed results (e.g., image data) via an input/output interface E/A. A non-transitory data storage medium 16 may be loaded into the control computer 9, and may be encoded with programming instructions (e.g., program code) that cause the control computer 9, and the various functional units thereof described above, to implement any or all embodiments of the method according to the present embodiments.

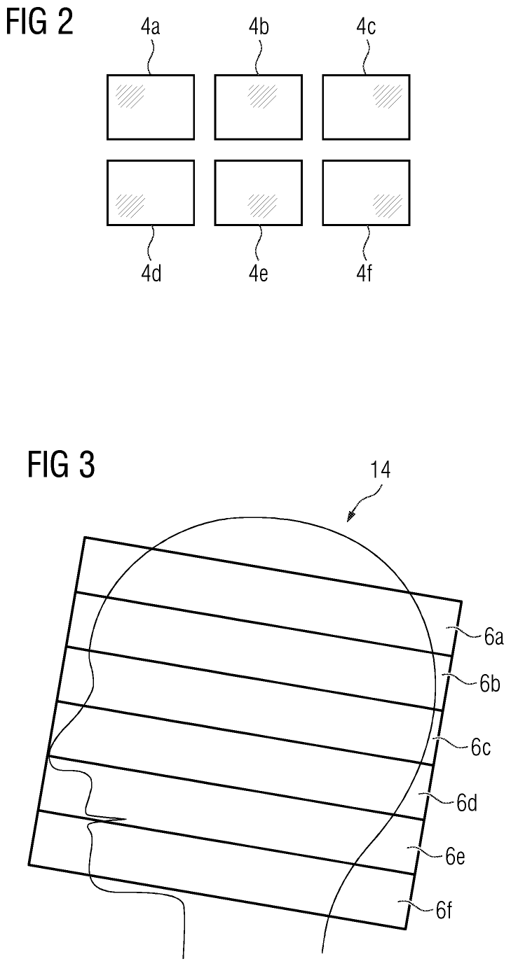

FIG. 2 illustrates a B1-map that was measured for a multi-element RF transmit coil 7 for an individual patient. In this embodiment, the transmit coil 7 has 6 channels (e.g., six coil elements. Each coil element has a different sensitivity throughout the body part to be measured. These RF sensitivity maps are illustrated at 4a, 4b, 4c, 4d, 4e and 4f for each individual element 7,1. 7,2 of the coil 7, and are together referred to herein as B1-map.

FIG. 3 illustrates a sagittal cross section through the human head 14, with six field-of-views 6a, 6b, 6c, 6d, 6e and 6f positioned therein in a typical almost transversal orientation. Each of these slabs or slices 6 may represent a field-of-view, for which a shim may be optimized. It is also possible that a field-of-view is only a part of a slice 6.

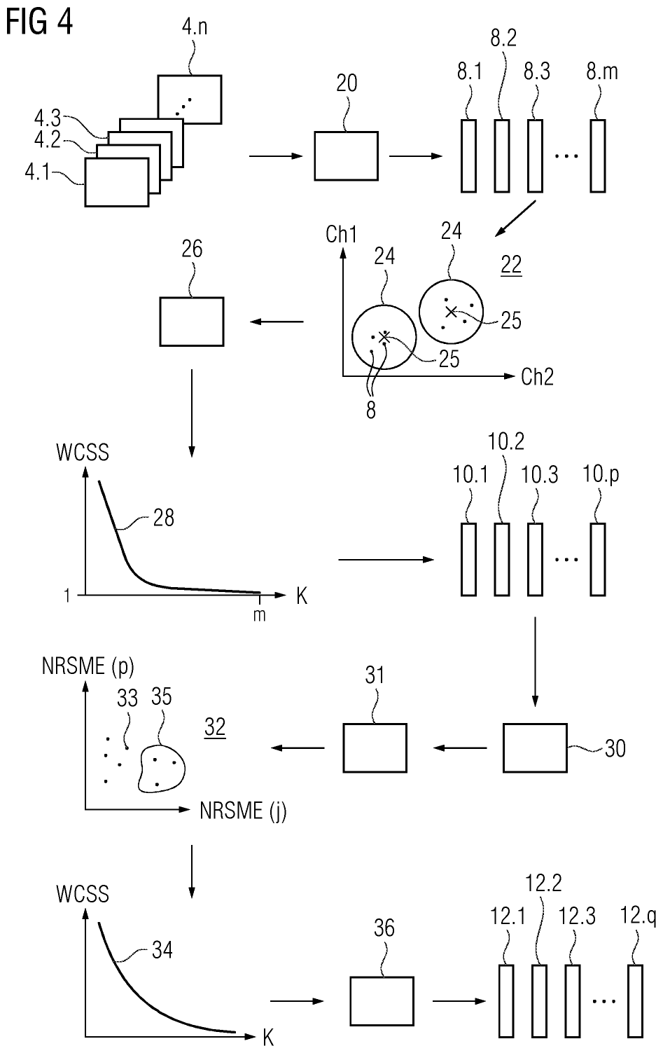

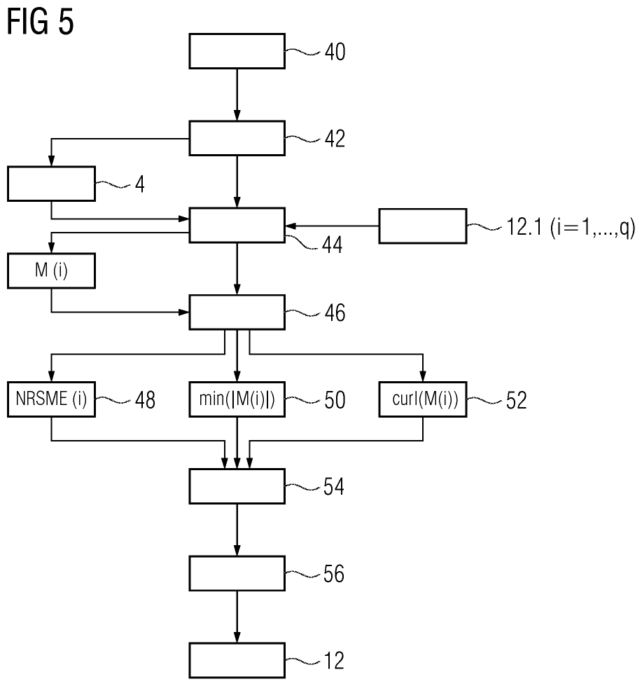

FIG. 4 illustrates a method according to an embodiment of the present embodiments. The optimization method, which may be executed online, requires as input a number n of B1-maps 4 acquired from various patients during different previous MR measurements. The number n of B1-maps 4 may be all from the same body part (e.g., all from the human head) or alternatively from a variety of body parts and are illustrated at 4.1, 4.2, 4.3, . . . , 4.n. In act 20, these different B1-maps 4 are used in order to optimize shims for a number m different field-of-views within these B1-maps, using a known optimization algorithm (e.g., a gradient descent method such as disclosed in Majewski). The result of this B1-shimming is a number m of B1-shims 8, where each B1-shim is essentially a vector of complex B1-shim coefficients. In one embodiment, B1-shims 8 are optimized for a number of field-of-views in a number of different orientations (e.g., transversal, coronal, sagittal). In one embodiment, circularly polarized B1-fields or shims are used as starting values.

The thereby obtained B1-shims 8 are then arranged in a first feature space illustrated at 22, where the coordinate axes of this feature space 22 are the real and imaginary parts of the shim coefficients, here illustrated as Ch1 (channel 1) and Ch2 (channel 2). In reality, each channel is represented by a complex shim coefficient, so there will be twice as many dimensions of this feature space 22 as channels or elements of the multi-element transmit coil 7. In the first feature space 22, each shim 8 is represented by one data point.

In act 26, k-means clustering is applied to the feature space 22 in order to divide the optimized B1-shims 8 into a number of clusters 24. In order to find a suitable number of clusters, the within-cluster-sum-of-squares (WCSS) value 28 may be calculated as a function of the number of clusters k, where the maximum number of clusters is m, the number of B1-shims. The WCSS value is a measure for how similar all B1-shims within one cluster are. Alternatively, the number of clusters may be predetermined. Once a suitable number of clusters has been defined, a clustering may be carried out to define which B1-shims belong to each cluster 24. In a next act, for each cluster 24, a center of gravity 25 (e.g., midpoint) is calculated. The centers of gravity 25 together may be regarded as covering a subspace of all possible B1-shims, in which possible useful B1-shims are contained. The centers of gravity 25 are then each regarded as a B1-shim by themselves, resulting in a set of average B1-shims 10.1, 10.2, 10.3, . . . , 10.p, where p is the number of clusters.

In act 30, these average B1-shims 10 are then used as starting points for an individual optimization on the various field-of-views, which have also been used in act 20. The result of this optimization 30 is then evaluated in act 31 by calculating the normalized root-mean-square deviations for each field-of-view. This may be done by simulating a flip angle distribution or B1-field distribution or magnetization distribution for each field-of-view in act 31, and comparing this with the target flip angle or B1-field (e.g., the magnitude thereof) or the magnetization (e.g., magnitude thereof). A suitable measure for the deviation may be the NRMSE. From these NRMSE values, a second feature space 32 is defined, in which each individual field-of-view is represented as one data point 33, and the coordinate axes or bases of this feature space 32 are the different NRMSE values of the different candidate shims 10. In this feature space, a further k-means clustering is carried out, where the different field-of-views are grouped into clusters 35. This may again be done by calculating the WCSS value for the number of clusters k, which may, for example, be 40. By this clustering, those field-of-views may be grouped together, for which a similar B1-shim works similarly well (or less well).

In a next act 36, a final B1-shimming/optimization is carried out, where the field-of-views for each cluster are grouped together (e.g., the B1-shim is optimized not for one field-of-view), but for a number of field-of-views simultaneously. This may be done for all field-of-views within one cluster, or only those that are closest to the center of gravity of the cluster 35, where the Euclidean distance may be used. The B1-shimming is done by using some or all of the B1-shims 8 (e.g., which had been used to define the first feature space) as starting value for the optimization, and again evaluating the quality of the resulting B1-shim (e.g., by calculating an NRMSE as described above). The B1-shim resulting from the optimization and having the lowest NRMSE for each group of field-of-views is then taken as one optimized initial B1-shims 12.

The different clusters 35 represent the different B1-field distributions to be optimized. By optimizing for a number of field-of-views (e.g., possibly in different orientations) together, more robust optimized initial B1-shims 12 may be found than in a purely individual optimization, in which “holes” in the magnetization distribution may more easily occur. The result of this optimization 36 is thus a set of optimized initial B1-shims 12, one for each cluster 35.

This method may be carried out for each individual magnetic resonance imaging system 1 (e.g., during its setup), and the set of optimized initial B1-shims 12 may be stored for later use during an MR measurement. Alternatively, they may be predefined for each type of MRI system 1 and multi-element RF coil 7.

FIG. 5 illustrates a method that may be carried out during an MR measurement to find the one optimized initial B1-shim 12 that is to be used as starting value for the B1-shimming in this particular examination. In act 40, the patient is placed in the sensitive area of the MRI system. In act 42, an individual B1-map 4 of the region of interest within the body part to be measured in this particular MRI measurement is acquired. The actual B1-map in this MRI measurement is used in act 44, as well as the set of optimized initial B1-shims 12.i, where i may be 1, 2, 3, . . . , q, which are the optimized initial B1-shims stored for this MRI system. In act 44, a magnetization matrix M (i) is calculated for each optimized initial B1-shim. In this calculation, a small angle approximation may be used for all flip angles. This is sufficient to evaluate the relevant differences between a number of shims and opens the possibility to define a matrix M for each field-of-view, and to then estimate the different B1-distributions for different B1-shims by a simple matrix multiplication. From these magnetization distributions, which are stored in the magnetization matrices M (i), three parameters are calculated in act 46, where the third parameter 52 is optional. These parameters are the NRMSE 48, the minimal flip angle 50, and the rotation 52. These two or three parameters are calculated for each field-of-view and added together in a weighted summation in act 54. In act ,56, the shim 12 with index i is identified, for which the weighted sum of these 3 terms is the smallest of all shims. This process may be represented by the following formula:

$${arg min}_{i} ( ( \frac{NRMSE ( i )}{{max}_{∀ i} ( NRMSE ( i ) )} ) + {w}_{minFA} ( 1 - \frac{min ( \vert M ( i ) \vert )}{{max}_{∀ i} ( min ( \vert M ( i ) \vert ) )} ) + {w}_{phase ro} \text{?} \frac{{\vert curl ( \begin{matrix}Re ( M ( i ) ) \\ lm ( M ( i ) )\end{matrix} ) \vert}_{1}}{{max}_{∀ i} {\vert curl ( \begin{matrix}Re ( M ( i ) ) \\ lm ( M ( i ) )\end{matrix} ) \vert}_{1}} )$$

$$\text{?} \text{indicates text missing or illegible when filed}$$

in which M represents the transverse magnetization in each voxel in the particular field-of-view as a matrix, equivalent to a B1-field distribution or flip angle plus phase, curl represents the rotation operator, and w_{minFA} is a weight for the minimal flip angle and may be between 0.5 and 2 (e.g., 1.2), and the weight for the phase rotation is w_{phase rod}, which may be between 1 and 2 (e.g., 1.8). Each of the three parameters is normalized (e.g., the NRMSE is divided by the maximum NRMSE of all shims with index i, so that the maximum value of this term is 1). In the second term, the minimum flip angle is divided by the minimum flip angle across all magnetization matrices with index i. In this case, the normalized minimum flip angle is subtracted from 1, so that this term is at a minimum if the minimum flip angle is at its maximum. In an embodiment, the minimum in each magnetization matrix is taken to be not the absolute minimum, but the minimum magnetization under exclusion of the lowest 1 to 5 (e.g., 2 percentiles in order to exclude possible outlying values). Also, the third term is normalized by dividing the rotation within each magnetization matrix by the maximum rotation across all magnetization matrices. The second parameter and also the third parameter is responsible for avoiding “holes” in the magnetization matrix and thus in the final image, since these often correlate with a strong phase rotation in the facility of the hole. The weight may be varied for different RF coils, and for the different body parts. The optimal weight parameters may also vary with the respective shim. The output of this method is one optimized initial B1-shim 12, which is then used to individually optimize the B1-shim for this particular field-of-view.

By the process in FIG. 5, one may very quickly find the optimal B1-shim for each field-of-view and use that as a starting value for the individual optimization. The time frame for finding the best initial B1-shim is mostly less than a second (e.g., between 0.1 and 1 seconds or between 0.2 and 0.8 seconds).

Therefore, the present embodiments provide a method to reach much improved B1-shims, and, for example, avoids “holes” in the final image. The processing is mostly done in an offline step, which may be carried out beforehand and only once for each multi-element RF coil. Possibly, a set of optimized initial B1-shims may be provided for different body parts in combination with a certain multi-element RF coil. During the actual MR measurement, after carrying out the selection method for selecting the best initial B1-shim, a state-of-the-art shimming optimization algorithm may be used, and still much improved B1-shimming results are obtained. This “cluster starting point” furnishes better homogeneity and higher flip angles, especially in those field-of-views in the lower part of the head, which are otherwise somewhat impaired by air inclusions in the throat area.

Independent of the grammatical term usage, individuals with male, female or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for calculating at least one optimized initial B1-shim for a magnetic resonance measurement, wherein a B1-shim comprises a vector of complex B1-shim coefficients, each coefficient of the vector of complex B1-shim coefficients representing a scaling factor for one element of a multi-element transmit coil that is to be used in the magnetic resonance measurement, the method comprising:
receiving a set of previously measured B1-maps of the multi-element transmit coil for one or more body parts of various test subjects;
calculating a set of B1-shims for a plurality of different field-of-views in the one or more body parts of the various test subjects from the previously measured B1-maps using an optimization algorithm;
identifying which B1-shim has a best performance for a group of field-of-views of the plurality of different field-of-views using the previously measured B1-maps; and
optimizing the B1-shim for the group of field-of-views, such that an optimized initial B1-shim is obtained.

2. The method of claim 1, wherein identifying which B1-shim has the best performance for the group of field-of-views using the previously measured B1-maps comprises:
clustering the B1-shims calculated for the plurality of different field-of-views and identifying an average B1-shim for each cluster;
applying the average B1-shims to each field-of-view of the plurality of different field-of-views and calculating a performance index for each combination of average B1-shim and field-of-view using the previously measured B1-maps; and
clustering the plurality of different field-of-views according to performance indices, such that groups of field-of-views that perform similarly well with a similar B1-shim are identified,
wherein the optimizing comprises optimizing the B1-shim for each of the groups of field-of-views, such that an optimized initial B1-shim is obtained.

3. The method of claim 1, further comprising:
performing a clustering of the calculated set of B1-shims and identifying an average B1-shim for each cluster, the clustering and identifying comprising:
representing the calculated set of B1-shims in a first feature space, wherein dimensions of the feature space are complex shim coefficients of each B1-shim; and
performing a cluster analysis of the B1-shims in the first feature space and calculating a midpoint of each cluster, each midpoint being the average B1-shim for the respective cluster.

4. The method of claim 3, further comprising:
clustering the plurality of different field-of-views according to the performance indices and optimizing the B1-shim, such that an optimized initial B1-shim is obtained for the group of field-of-views, the clustering of the plurality of different field-of-views according to the performance indices and the optimizing comprising:
representing the calculated performance indices in a second feature space, in which each field-of-view of the plurality of different field-of-views is represented by one data point, and dimensions of the second feature space are the performance indices of each average B1-shim;
performing a cluster analysis on the second feature space, and determining the groups of field-of-views that are closest to a center of each cluster;
for each group of field-of-views, calculating an optimized B1-shim using an optimization algorithm; and
providing the optimized B1-shims as optimized initial B1-shims for the field-of-views within the group of field-of-views.

5. The method of claim 4, wherein the clustering of the B1-shims in the first feature space, the clustering of the field-of-views in the second feature space, or the clustering of the B1-shims in the first feature space and the clustering of the field-of-views in the second feature space are performed using a k-means Clustering algorithm.

6. The method of claim 2, wherein calculating the performance index for each combination of average B1-shim and field-of-view comprises simulating a magnetization distribution or flip angle distribution from each B1-shim and comparing the simulated distribution with a target magnetization or flip angle distribution.

7. The method of claim 6, wherein comparing the simulated distribution with the target magnetization or flip angle distribution comprises calculating a root-mean-square deviation.

8. The method of claim 1, further comprising calculating a set of optimized initial B1-shims.

9. The method of claim 1, further comprising calculating one optimized initial B1-shim for each group of field-of-views.

10. The method of claim 1, further comprising a shimming method for performing B1-shimming during a magnetic resonance measurement on a field-of-view within a body part of a subject using a multi-element transmit coil, the shimming method comprising:
receiving a set of optimized initial B1-shims that have been calculated by the method for calculating at least one optimized initial B1-shim;
measuring B1-maps of the body part;
calculating a magnetization distribution resulting from the combination of each of the set of optimized initial B1-shims with the field-of-view using the measured B1-maps and storing the calculated magnetization distribution in a magnetization matrix;
for each of the set of optimized initial B1-shims, calculating a term, the term comprising a parameter, the parameter comprising a comparison of the magnetization matrix with a target magnetization distribution, and calculating a parameter comprising a minimal magnetization or flip angle within the magnetization matrix; and
selecting the optimized initial B1-shim for which the calculated term is at an extremum as a starting point of a B1-shimming optimization.

11. The method of claim 10, wherein the calculated term comprises a parameter comprising a phase rotation of the magnetization matrix.

12. The method of claim 10, wherein the calculated term comprises a parameter comprising a root-mean-square deviation between the magnetization matrix and a target magnetization distribution.

13. The method of claim 12, wherein the calculated term is a weighted sum of a parameter comprising the root-mean-square deviation between the magnetization distribution and the target magnetization distribution, and a parameter including the minimum flip angle within the magnetization matrix.

14. The method of claim 12, wherein the calculated term is a weighted sum of a parameter comprising the root-mean-square deviation between the magnetization distribution and a target magnetization distribution, a parameter including the minimum flip angle within the magnetization matrix, and a parameter including a phase rotation of the magnetization matrix.

15. A non-transient computer-readable storage medium that stores instructions executable by one or more processors to calculate at least one optimized initial B1-shim for a magnetic resonance measurement, wherein a B1-shim comprises a vector of complex B1-shim coefficients, each coefficient of the vector of complex B1-shim coefficients representing a scaling factor for one element of a multi-element transmit coil that is to be used in the magnetic resonance measurement, the instructions comprising:
receiving a set of previously measured B1-maps of the multi-element transmit coil for one or more body parts of various test subjects;
calculating a set of B1-shims for a plurality of different field-of-views in the one or more body parts of the various test subjects from the previously measured B1-maps using an optimization algorithm;
identifying which B1-shim has a best performance for a group of field-of-views using the previously measured B1-maps; and
optimizing the B1-shim for the group of field-of-views, such that an optimized initial B1-shim is obtained.

16. A B1-shim design unit configured to calculate at least one optimized initial B1-shim for a magnetic resonance imaging measurement on a field-of-view within a body part of a subject, wherein a B1-shim comprises a vector of complex B1-shim coefficients, each coefficient of the vector of complex B1-shim coefficients representing a scaling factor for one element of a multi-element transmit coil that is to be used in the magnetic resonance measurement, the B1-shim design unit comprising:
a data interface configured to:
receive a set of previously measured B1-maps of the multi-element transmit coil for one or more body parts of various test subjects; and
output the at least one optimized initial B1-shim; and
a processor configured to:
calculate a set of B1-shims for a plurality of different field-of-views in the one or more body parts of the various test subjects from the previously measured B1-maps using an optimization algorithm;
identify which B1-shim has a best performance for a group of field-of-views using the previously measured B1-maps; and
optimize the B1-shim for the group of field-of-views, such that an optimized initial B1-shim is obtained.

17. A control unit for a magnetic resonance imaging system, the control unit comprising:
a processor configured to:
calculate at least one optimized initial B1-shim for a magnetic resonance measurement, wherein a B1-shim comprises a vector of complex B1-shim coefficients, each coefficient of the vector of complex B1-shim coefficients representing a scaling factor for one element of a multi-element transmit coil that is to be used in the magnetic resonance measurement, the processor being configured to calculate at least one optimized initial B1-shim for the magnetic resonance measurement comprising the processor being configured to:
receive a set of previously measured B1-maps of the multi-element transmit coil for one or more body parts of various test subjects;
calculate a set of B1-shims for a plurality of different field-of-views in the one or more body parts of the various test subjects from the previously measured B1-maps using an optimization algorithm;
identify which B1-shim has a best performance for a group of field-of-views of the plurality of different field-of-views using the previously measured B1-maps; and
optimize the B1-shim for the group of field-of-views, such that an optimized initial B1-shim is obtained.

18. A magnetic resonance imaging system comprising:
a control unit for a magnetic resonance imaging system, the control unit comprising:
a processor configured to:
calculate at least one optimized initial B1-shim for a magnetic resonance measurement, wherein a B1-shim comprises a vector of complex B1-shim coefficients, each coefficient of the vector of complex B1-shim coefficients representing a scaling factor for one element of a multi-element transmit coil that is to be used in the magnetic resonance measurement, the processor being configured to calculate at least one optimized initial B1-shim for the magnetic resonance measurement comprising the processor being configured to:
receive a set of previously measured B1-maps of the multi-element transmit coil for one or more body parts of various test subjects;
calculate a set of B1-shims for a plurality of different field-of-views in the one or more body parts of the various test subjects from the previously measured B1-maps using an optimization algorithm;
identify which B1-shim has a best performance for a group of field-of-views of the plurality of different field-of-views using the previously measured B1-maps; and
optimize the B1-shim for the group of field-of-views, such that an optimized initial B1-shim is obtained .
